(19) United States
(12) Patent Application Publication
(10) Pub. No.: US 2025/0025634 A1
(43) Pub. Date: Jan. 23, 2025

(54) INJECTION DEVICE AND METHOD OF UNLOCKING OPERATION OF THE INJECTION DEVICE

(71) Applicant: Sanofi, Paris (FR)

(72) Inventors: Stefan Alt, Frankfurt am Main (DE); Michael Helmer, Frankfurt am Main (DE); Michael Schabbach, Frankfurt am Main (DE); Martin Vitt, Frankfurt am Main (DE)

(21) Appl. No.: 18/706,818

(22) PCT Filed: Nov. 1, 2022
(86) PCT No.: PCT/EP2022/080413

§ 371 Date: May 2, 2024

(30) Foreign Application Priority Data
Nov. 3, 2021 (EP) 21315208.5

(51) Int. Cl. A61M 5/315

(52) U.S. Cl. CPC A61M 5/31501; A61M 5/31586; A61M 2205/3569; A61M 2205/6009

(57) ABSTRACT

The present disclosure relates unlocking operation of an injection device. The injection device includes a housing configured to accommodate a medicament container and a drive mechanism operably engageable with the medicament container for expelling or withdrawing the dose of the medicament, where the drive mechanism includes at least one interlock configured to disable at least one of setting of the dose and dispensing of the dose. The injection device further includes an identifier provided on or associated with the medicament container and an electronic unit operably engaged with the at least one interlock, the electronic unit including a short-range communication interface to communicate with a corresponding short range communication interface of an external electronic device to obtain information indicative of the identifier. The electronic unit is configured to process signals of the short-range communication interface to at least temporally deactivate the at least one interlock.

### CROSS REFERENCE TO RELATED APPLICATIONS

The present application is the national stage entry of International Patent Application No. PCT/EP2022/080413, filed on Nov. 1, 2022, and claims priority to Application No. EP 21315208.5, filed on Nov. 3, 2021, the disclosures of which are incorporated herein by reference.

### TECHNICAL FIELD

The present disclosure relates to an injection device, such as a pen-type injector and to a method of unlocking operation of the injection device, e.g., by using an external electronic device. In further aspects, the disclosure relates to an electronic device and to an injection system or drug delivery system as well as to a computer readable medium that implement unlocking of the injection device.

### BACKGROUND

Drug delivery devices for setting and dispensing a single or multiple doses of a liquid medicament are used for various purposes. Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore tend to be robust in construction and easy to use. Furthermore, manipulation and general handling of the device and components thereof should be intelligible and easily understood. It may be beneficial for such injection devices to provide setting and subsequent dispensing of a dose of a medicament of variable size. Moreover, a dose setting as well as a dose dispensing procedure should generally be easy to operate and unambiguous.

A patient suffering from a particular disease may require a certain amount of a medicament, which can either be injected via a pen-type injection syringe or infused via a pump.

Some drug delivery or injection devices provide selecting of a dose of a medicament of variable size and injecting a dose previously set. Other injection devices provide setting and dispensing of a fixed dose. In such cases, the amount of medicament that should be injected in accordance with a given prescription schedule does not change (or cannot be changed) over time.

Some injection devices are implemented as a reusable injection device with a replaceable medicament container, such as a cartridge. Other injection devices are implemented as a disposable injection device. With disposable injection devices, the entirety of the injection device is discarded when the content (e.g., the medicament) has been used.

With reusable injection devices a medicament container, e.g., a cartridge has to be replaced when the medicament has been depleted. Here, the medicament container, e.g., a vitreous cartridge filled with the medicament has to be replaced. Some primary medicament containers, e.g., in the form of a cartridge filled with the medicament are preassembled in a housing component of the injection device, such as a cartridge holder.

For certain reusable devices and disposable injection devices, only a dedicated medicament container or dedicated housing component preassembled with such a medicament container can be used with a dedicated drive mechanism of the injection device.

Generally, there is a large variety of primary medicament containers that differ with regard to their filling volume or pharmaceutical substance or concentration of the medicament. For replacement of a medicament container, an injection device or drive mechanism can only be used in conjunction with a dedicated medicament container intended for use with this particular type of injection device or drive mechanism. Unintended cross-use of non-matching pairs of medicament containers and drive mechanisms or injection devices should be avoided.

To prevent unintended mechanical connection to a non-matching drive mechanism or non-matching injection device, some approaches involve mechanically encoding the primary container, e.g., the medicament container and/or the cartridge holder equipped with such a medicament container. Such solutions typically involve a redesign of respective mechanical components of the injection device. Providing a large variety of mechanically encoded housing components can increase the manufacturing costs of the injection device.

### SUMMARY

This disclosure relates to an improved injection device, an electronic device, an injection system, a method of operating an injection device, and a computer readable medium to enhance patient safety and to prevent unintended cross use of replaceable medicament containers with an injection device. The present disclosure aims to avoid a mechanical redesign of housing components of existing injection devices. It is desirable to provide a solution that is easily integrable into existing injection devices or injection systems and easily adaptable to varying demands or varying scenarios of use of the injection device.

In one aspect, the present disclosure relates to an injection device for injecting of a dose of a medicament. The injection device includes a housing configured to accommodate a medicament container. The injection device further includes a drive mechanism operably engageable with the medicament container for expelling or withdrawing the dose of the medicament from the medicament container. The drive mechanism includes at least one interlock configured to disable at least one of setting of the dose and dispensing of the dose of the medicament. With an activated interlock at least one of setting of the dose and dispensing of the dose is disabled. With a disabled interlock, setting and/or dispensing of the dose is enabled.

There is further provided an identifier on the medicament container or associated with the medicament container. Furthermore, the injection device includes an electronic unit. The electronic unit or electronic module is operable to control the at least one interlock. The injection device further includes a short-range communication interface to communicate with a corresponding short-range communication interface of an external electronic device for obtaining information being indicative of the identifier or data provided by the identifier. The electronic unit is further configured to process signals of the short-range communication interface to at least temporarily deactivate the at least one interlock.

In some implementations, the electronic unit and/or the short-range communication interface is or are operable to read information directly from the identifier or to communicate with the identifier when the identifier is provided on the medicament container or when the identifier is associated with the medicament container.

Here, with the help of the external electronic device, information being indicative of the identifier and/or identifier information being indicative of the medicament container can be captured, read and/or processed. Based on the identifier related information retrievable by the external electronic device respective signals can be transmitted from the external electronic device to the short-range communication interface of the electronic unit of the injection device. Based on signals obtained from the external electronic device and based on signals of the short-range communication interface as derived from signals obtained from the external electronic device the at least one interlock can be at least temporally deactivated.

When the information or data which is indicative of the identifier matches with predefined medicament container related data the respective medicament container can be used with the drive mechanism of the injection device.

In some embodiments, wherein the identifier does not match with the drive mechanism and wherein the information obtained by the external electronic device from the identifier and communicated via the short-range communication interface to the electronic unit, does not match with predefined medicament container related data the interlock remains activated and use of the injection device, e.g., for the purpose of setting and/or dispensing of the dose is impeded or blocked.

Making use of an external electronic device as a kind of an electronic relay to provide at least one of acquiring, capturing, obtaining, or retrieving data from the identifier provides a large variety of possible technical implementations of such identifiers on or associated with the medicament container. This way, communication capabilities or communication hardware of the electronic unit does not necessarily have to match with the technical implementation of the identifier. Instead, the external electronic device can be used as a data transmitter or data converter. Here, the external electronic device is enabled to cooperate with both, the identifier provided on or associated with the medicament container and the short-range communication interface of the electronic unit of the injection device.

In this way, the identifier can be implemented as an electronic identifier, as an electronic passive identifier, as an optical or visual identifier or the like. The identifier cooperates and matches with a communication interface or sensor of the external electronic device, which in turn is capable to communicate with the electronic unit of the injection device via the short-range communication interface.

In some examples, the external electronic device is implemented as a smartphone, as a smart watch or as a tablet computer that is enabled to read or to capture the identifier. Furthermore, the electronic device is capable to communicate, e.g., to wirelessly communicate with the short-range communication interface of the electronic unit of the injection device. In this way, the electronic unit can be embedded in or on the injection device. The electronic device may be arranged inside the housing of the injection device and may be permanently connected to or assembled inside the injection device. In some embodiments, the electronic unit is an embedded electronic unit being integrated into the injection device or the drive mechanism of the injection device.

In such examples, establishing of a direct communication link between the identifier provided on or associated with the medicament container and the electronic unit and/or the short-range communication interface of the electronic unit may be complicated. In some embodiments setting up a communication link with the short-range communication interface uses an electrical power source for both a transmitter and a receiver. With the currently proposed solution of an identifier providing information that is retrievable, capturable, or obtainable via the external electronic device there is no need to provide the identifier with an electric energy supply. This enables to implement the identifier as a passive component, e.g., an electric or electronic component which is unpowered.

The external electronic device may be implemented in various different ways. With one example the external electronic device is able to read or to capture the identifier and to transmit the identifier information to the electronic unit via the communication link between the corresponding short-range communication interface of the external electronic device and the short-range communication interface of the electronic unit of the injection device.

Signal processing may be then exclusively conducted by the electronic unit of the injection device in order to keep the interlock activated, e.g., when the identifier does not match with a predefined requirements, demand or scenario of use. The electronic unit may keep the at least one interlock activated so that at least one of setting and dispensing of the dose is and remains disabled. When the data obtained from the identifier matches predefined requirements, demands or scenarios of use the electronic unit may at least temporally deactivate the at least one interlock and the drive mechanism becomes unlocked for providing at least one of setting and injecting the dose of the medicament.

When processing of the data is conducted by the processor of the electronic unit of the injection device, the external electronic device does not have to provide any kind of a data processing. The electronic device may simply read or obtain the data or information from the identifier and may unalterably transfer or transmit the respective information to the electronic unit. With this approach, there is comparatively less software or hardware adaptation for the external electronic device.

With other examples, a processing of information retrieved from the identifier may be conducted by the external electronic device. Then, the external electronic device may be operable to compare the data obtained from the identifier with predefined requirements, demands or scenarios of use of the injection device. For this, the external electronic device may be provided with a respective device specific software or computer program, e.g., an App.

When the data obtained from the identifier matches predefined demands, requirements or scenarios of use, the external electronic device may unlock the at least one interlock via the mutually communicating short-range communication interfaces. With this solution, the external electronic device has been previously authenticated to control the at least one interlock of the electronic unit.

According to a further example, the housing of the injection device includes a longitudinal structure that extends along a longitudinal direction. The housing may include a distal housing component at a proximal housing component. The distal housing component, also denoted as cartridge holder, is configured to accommodate the medicament container. The proximal housing component is configured to accommodate or to support the drive mechanism. The distal housing component includes a distal end facing away the proximal housing component. The distal end of the distal housing component is provided or connected with an injection needle, so as to pierce or to puncture biological tissue for injecting the medicament.

The proximal end of the distal housing component is detachably connectable to a distal end of the proximal housing component. By detaching or disconnecting the distal housing component from the proximal housing component the medicament container, e.g., a cartridge filled with the medicament, can be replaced on demand.

Moreover, the drive mechanism provided on or in the proximal housing component is resettable. When the housing components are disconnected, the drive mechanism may be subject to a reset operation so that the injection device can be set into an initial configuration after inserting a new medicament container into the distal housing component and upon reconnecting the distal housing component with the proximal housing component.

According to a further example, the identifier is located on or inside the distal housing component. The electronic unit is provided in or on the proximal housing component. In some embodiments the electronic unit and/or the at least one interlock is mechanically engaged with at least one movable component of the drive mechanism. The electronic unit may be provided inside the proximal housing component. The electronic unit may be provided or arranged at or near a proximal end of the proximal housing component. With the identifier located on or inside the distal housing component the identifier may be located remote from the electronic unit and/or remote from the short-range communication interface of the electronic unit.

In some embodiments, the identifier is located on the medicament container. With other examples the identifier is located on the distal housing component, wherein the distal housing component is preassembled with the medicament container. Here, the medicament container may be non-detachably connected to the distal housing component. In some embodiments the distal housing component is detachably connectable and/or detachably fixable to the proximal housing component.

According to a further example, the identifier includes at least one of an optical code and a visible code. The optical code or visible code can be captured by the external electronic device. Here, the external electronic device including at least one optical sensor. The external electronic device may be equipped with a two-dimensional sensor array. The external electronic device may be provided with an imaging sensor so as to capture a two-or one-dimensional image of the optical code. In some embodiments the external electronic device is equipped with a camera to capture an image of the optical or visual code.

The external electronic device may be further configured to extract code information or medicament related information from the captured optical or visible code and to provide or to transmit the extracted data or information to the electronic unit via the communication link between the short-range communication interface of the electronic unit and the corresponding short-range communication interface of the external electronic device.

With the external electronic device, it is possible to assemble the distal and proximal housing component with the medicament container located inside the housing of the injection device and to subsequently conduct a validation or approval routine of the medicament container by the identifier, the external electronic device and the short-range communication between the external electronic device and the electronic unit so as to at least temporally deactivate the interlock of the injection device.

In a further example, the identifier includes a passive electronic identifier. A passive electronic identifier may electronically communicate with the external electronic device. The passive electronic identifier may be incompatible with the short-range communication interface of the electronic unit of the injection device. Such an incompatibility can be bridged or bypassed by the external electronic device operable to read the identifier and operable to communicate with the short-range communication interface of the electronic unit of the injection device.

According to a further example, the passive electronic identifier is an unpowered electronic identifier being void of a permanent source of electric energy. This allows to reduce manufacturing costs and to save storage space for implementing the identifier on the medicament container or on the distal housing component. A passive or unpowered electronic identifier can be manufactured in larger quantities at lower costs. The identifier can be implemented or configured as a disposable electronic identifier.

According to a further example, the passive electronic identifier includes a microchip and an antenna. The antenna is configured to harvest electric energy from a surrounding electromagnetic field. In some embodiments, the passive electronic identifier is implemented as a near-field communication (NFC) tag. In some embodiments, the passive electronic identifier includes a passive RFID tag. Furthermore, and In some embodiments, the passive electronic identifier includes a NFC tag configured to communicate with a corresponding NFC interface of the external electronic device. Here, the external electronic device includes two communication interfaces, namely a NFC interface to communicate with the passive electronic identifier provided on or associated with the medicament container. The external electronic device further includes the above-mentioned short-range communication interface to communicate with the short-range communication interface of the electronic unit of the injection device.

When the identifier is implemented as a NFC tag, e.g., as an NFC tag or RFID tag, the identifier is primarily inoperable to communicate with the short-range communication interface of the electronic unit of the injection device. The electronic unit of the injection device may be void of a NFC interface that would be suitable for reading or capturing data from the NFC tag of the identifier.

In some embodiments, the external electronic device is compatible with both, the NFC interface and the short-range communication interface and provide a relay function so as to capture or to obtain information from the electronic identifier and to transmit the respective information to the electronic unit via the short-range communication interfaces.

The communication link between the external electronic device and the passive electronic identifier may have bidirectional type. Hence, the external electronic device may not be limited to reading or to capturing information from the passive electronic identifier, but may also be operable to transmit data to the passive electronic identifier and/or to store or to modify a nonvolatile memory of the passive electronic identifier.

In this way, once the passive electronic identifier has been read out by the NFC interface of the external electronic device, the passive electronic identifier of the injection device may be invalidated. Here, the passive electronic identifier may be electronically marked as being in use or that the associated medicament container has been used and cannot be used any further, e.g., with another injection device.

According to a further example, the electronic identifier includes a nonvolatile memory to store at least one of a medicament name, a medicament manufacturing date, a medicament expiry date, a LOT number of the medicament or a unique medicament container ID. In this way, a relatively large variety of medicament related data can be stored in or by the identifier and can be captured or retrieved through a readout of the identifier by the external electronic device.

The above-mentioned medicament-related parameters, such as medicament name, manufacturing date, expiry date, LOT number or a unique medicament container ID may be also encoded in an optical or visual code to be captured by a camera of the external electronic device. In some embodiments, the visible code may include a QR code or a data matrix code which can be translated into respective data via a network connection with a respective decoder.

According to a further example, the nonvolatile memory of the electronic identifier is at least one of erasable or writable by the NFC interface of the external electronic device. In this way, the nonvolatile memory of the passive electronic identifier can be reconfigured. There may be provided a validation flag in the nonvolatile memory indicating if the respective electronic identifier has been subject to a read or write operation before. By providing an erasable or writable nonvolatile memory, such a read or invalidation flag can be set or reconfigured upon a first reading or readout of the nonvolatile memory by the NFC interface of the external electronic device.

In this way, patient safety can be enhanced so as to prevent a multiple sequential use of the same medicament container, e.g., with the same or with different injection devices. In particular, a medicament container provided with a unique medicament container ID can be marked to be invalidated, so that an abuse of the container, e.g., a non-intended refilling of the container becomes less effective because then, such a counterfeited container will be inoperable to deactivate the interlock of the injection device.

With a further example, the short-range communication interface of the electronic unit includes a wireless communication interface based on one of the following communication standards, Bluetooth, Bluetooth low energy (BLE), ZigBee or Wi-Fi. In some embodiments the short-range communication interface of the electronic unit and the corresponding short-range communication interface of the external electronic device includes a transmission range that is larger than a transmission range of the NFC between the NFC tag of the identifier and the communication interface of the external electronic device.

In some embodiments, the spatial distance between the identifier provided on the medicament container or the distal housing component and the electronic unit of the injection device is larger than the transmission range of the NFC interface. Therefore, providing the electronic unit of the injection device with a NFC interface would be somewhat useless because the medicament container provided with the respective passive electronic identifier assembled inside the housing would be out of the transmission range of the NFC interface. Here, the external electronic device, implemented as a portable electronic device, provides a relay function so as to enable an indirect communication between the electronic identifier provided on or associated with the medicament container and the electronic unit, e.g., provided at the proximal end of the housing of the injection device.

According to a further example, the at least one interlock of the drive mechanism includes an electromechanical interlock configured to mechanically engage with a first component of the injection device and with a second component of the injection device. The first component is movable relative to the second component for at least one of setting of the dose and injecting of the dose of the medicament. The at least one interlock is operable to switch between an interlocked state, in which the first component is movably locked to the second component and a release state, in which the first component is movable relative to the second component, for at least one of setting of the dose and dispensing or injecting of the dose.

In some embodiments, the first component is movable in longitudinal direction relative to the second component for at least one of setting of the dose and injecting of the dose. In other examples, the first component is rotatable relative to the second component for at least one of setting of the dose and injecting of the dose. With a rotatable relative displacement between the first component and the second component, an axis of rotation extends along the longitudinal direction or axial direction of the housing of the injection device.

In some embodiments, the first component is subject to a helical motion relative to the second component for setting and/or injecting of the dose of the medicament. At least one of the first and the second components may belong to the drive mechanism. In some embodiments, both, the first and the second components belong to the drive mechanism of the injection device. With other examples at least one of the first and the second component is or forms part of the housing of the injection device.

In some embodiments and with a further example the electromechanical interlock includes an electrically implemented actuator operably engaged with a latch. In some embodiments, the latch is movable by the actuator along a longitudinal direction, along a radial direction and/or along a tangential direction with regards to a cylindrical or tubular geometry of the housing of the injection device. In some embodiments the actuator is located and/or fixed on one of the first and second components. The latch may be provided on the same component and may be mechanically engageable with the other one of the first and second component in order to block or to impede a relative movement of the first component and the second component when the interlock is in the interlocked state.

In some embodiments, the drive mechanism of the injection device includes a piston rod, a number sleeve, a drive sleeve or driver and at least one of a dose dial and a trigger or dose button. In some embodiments for setting of a dose the dose dial is rotatable relative to the housing. During dose setting the rotational movement of the dial is transferred to a respective rotational movement of the number sleeve. Here, and for impeding setting of a dose, at least one of the dose dials and the number sleeve may be rotationally and/or translationally locked by the interlock.

For dispensing or injecting of the dose the trigger or dose button has to be depressed by a user. The trigger button may be depressible relative to the dose dial, relative to the housing, relative to the number sleeve or relative to the piston rod. During dose dispensing the driver or drive sleeve may be subject to a rotational movement that is transferred into a longitudinal and/or rotational movement of the piston rod. During dose injection, the number sleeve typically rotates in a direction opposite to a direction of rotation during setting of the dose. Here, the interlock may be operable to block actuation or movement of the trigger, to block a rotation of the driver or drive sleeve, to block a rotation or movement of the piston rod and/or to block or to inhibit rotation or movement of the number sleeve.

Apart from these examples, further implementations of the interlock or electromechanical interlock are possible and conceivable as long as the first and second component to be mutually locked and engaged are subject to a relative movement during at least one of setting of the dose and dispensing of the dose.

According to a further example, the drive mechanism includes at least one clutch operable to switch the drive mechanism between a dose setting mode and a dose injecting or dose dispensing mode. The at least one interlock is operably engaged with the at least one clutch to selectively disable switching of the drive mechanism from one of the dose setting mode and the dose injecting mode into the other one of the dose setting mode and the dose injecting mode.

In some embodiments, the drive mechanism includes a first clutch and a second clutch. By way of a first clutch the number sleeve can be selectively engaged with a dose dial, a driver or drive sleeve of the drive mechanism. In this way, a rotation of the dose dial can be transferred into a respective rotation of the number sleeve during setting of the dose. During injecting of the dose, the number sleeve can be decoupled and released from the dose dial. Here, the number sleeve is allowed to rotate in an opposite sense of rotation while the dose dial does not have to follow this rotation but remains rotationally locked to the housing of the injection device.

By way of a second clutch, the dose dial and/or the number sleeve can be selectively engaged with a driver or drive sleeve of the drive mechanism. Furthermore, by way of the second clutch and/or by way of a third clutch, there may be a selective engagement between a driver or drive sleeve and the piston rod so as to move the piston rod in distal direction exclusively during injecting of the dose.

The drive mechanism of the injection device can be implemented in various different ways. One example of a drive mechanism is disclosed in WO 2004/078239 A1, the entirety of which is incorporated herein by reference. Another example of a drive mechanism to be implemented with the injection device is disclosed in WO 2014/033195 A1, the entirety of which is incorporated herein by reference.

According to a further example, the at least one interlock is controlled by a processor of the electronic unit. The processor is configured to activate the at least one interlock if one or more of the following conditions have been detected by the electronic unit. Such conditions may include: end of a dose injection, depletion of the medicament container, lapse of a predefined time interval since deactivation of the at least one interlock, removal of the medicament container from the housing, insertion of the medicament container into the housing, conducting of a manual reset of the drive mechanism or abrogation of a communication link between the short-range communication interface with the external electronic device.

There may be other conditions by way of which the processor is triggered to activate or to de-activate the interlock. In some embodiments and with any of the above conditions an automated activation of the interlock can be provided, thereby effectively preventing unintended or non-authorized use of the injection device. Any of the above-mentioned conditions can be detected e.g., by at least one sensor as described further below

According to a further example, the injection device includes the medicament container filled with the medicament. The medicament container is arranged inside the housing. In some embodiments the medicament container includes a cartridge filled with the medicament. The cartridge may include a tubular-shaped barrel, e.g., made of a vitreous material. The barrel may be sealed towards the proximal direction by a stopper movable along the longitudinal direction of the barrel for expelling the liquid medicament through a distally located outlet of the barrel.

The distally located outlet may be sealed by a seal. The seal at the distal end of the cartridge may include a sealing disc, e.g., made of a rubber material and being pierceable by a double-tipped injection needle. The rubber seal may be fixed or assembled to the distal end of the tubular barrel by way of a crimped metal cap.

According to a further example, the medicament container is non-removably attached to the distal housing component. The distal housing component includes a tubular-shaped cartridge holder shaped and configured to accommodate a medicament cartridge therein. The cartridge holder may be provided with a snap feature in order to fix the cartridge inside the cartridge holder. In this way, there may be provided a cartridge holder cartridge pre-assembly that may be commercially distributed to end consumers or patients. With such a preassembly the identifier may be provided on an outside surface of the distal housing component, hence on an outside surface of the cartridge holder.

In other examples, the identifier is provided on an outside surface of the barrel of the medicament container. With other examples the identifier is provided on a stopper of the cartridge or inside the stopper of the cartridge.

When the identifier is located on or in the medicament container, the identifier may be readable by the external electronic device even when the cartridge or medicament container is located at/or assembled inside the distal housing component. For this there may be provided a window or an aperture in a sidewall of the distal housing component. Alternatively, or additionally, the sidewall of the distal housing component is transparent or translucent.

In other implementations, e.g., when the identifier is implemented as a passive electronic identifier, the sidewall of the distal housing component may be permeable for electromagnetic radiation by way of which the passive electronic identifier can be read out.

According to a further example, the electronic unit of the injection device includes at least one sensor to detect or to measure movement of at least one movable component of the injection device. The movable component of the injection device may be indicative of a particular device configuration or device operation. By detecting the movement or the magnitude of the movement of the movable component the electronic unit is provided with information regarding the operational status of the injection device.

In some embodiments, the sensor is operable to quantitatively measure the degree of movement of the at least one movable component. The at least one sensor may be operable to measure a degree of rotation and/or a degree of longitudinal movement of the at least one movable component, e.g., relative to the housing of the injection device or relative to another movable component of the drive mechanism of the injection device.

By way of the at least one sensor there may be derived data with regards to a dose size. Here, the at least one sensor is operable to provide electrical signals being indicative of a size of a dose actually set and/or dispensed or injected by the drive mechanism of the injection device. In some embodiments the electronic unit is provided with a memory as well as with a clock. In this way a dosing history or injection history can be automatically captured and stored in the memory of the electronic unit.

By way of the short-range communication interface and by way of a communication link with an external electronic device dispensing-and hence injection-related data, such as an injection history can be synchronized with the external electronic device and can be transmitted to medical service providers for further data analysis. In this way, repeated use of the injection device can be precisely tracked and measured and the user's compliance with a given prescription schedule can be monitored.

According to a further aspect, the present disclosure relates to a kit of injection devices, in particular to an injection device of a first type and at least to an injection device of a further type, e.g., a second type. Injection devices of the first type and of the second type distinguish by their medicament or medicament container. The medicament container of the first type may distinguish from a medicament container of the second type by the size, the type of medicament, the pharmaceutically active substance and/or by the concentration of the pharmaceutically active substance.

The medicament container of the first type is exclusively configured for use with a drive mechanism of a first type. The medicament container of the second type is exclusively configured for use with a drive mechanism of a second type. Unintended cross use, e.g., use of a medicament container of the first type with an injection mechanism of the second type should be avoided.

For this purpose, the medicament container of the first type is equipped and/or associated with an identifier of the first type. The medicament container of the second type is equipped and/or associated with an identifier of the second type. By way of the identifier the respective type of the medicament container can be detected. Here, the identifier provides a coding. The identifier of the first type represents a first coding. The identifier of the second type represents a second coding. The identifiers of different types are processable and/or detectable by the electronic unit so as to activate or to deactivate the interlock.

The identifiers may represent an electronic coding or a coding that is electronically detectable. Such identifiers or codings enable use of mechanically unencoded housing components for the injection devices of the first and the second type. Even though a distal housing component equipped with a medicament container of the first type is assembled with a proximal housing component of a second type and equipped with a second type of an injection device such a combination will not be operable to abrogate or to deactivate the interlock of the drive mechanism. Setting and/or injecting of a dose will be effectively impeded.

In some embodiments, the electronic unit of the injection device is inoperable to gather or to read information of the identifier. The short-range communication interface of the electronic unit may be inoperable to communicate with the identifier or may be inoperable to read the identifier.

The identifier may cooperate and/or match with a communication interface or sensor of the external electronic device, which in turn is capable to communicate with the electronic unit of the injection device via the short-range communication interface.

This way, communication capabilities or communication hardware of the electronic unit does not necessarily have to match with the technical implementation of the identifier. Instead, the external electronic device can be used as a data transmitter or data converter. Here, the external electronic device is enabled to cooperate with both, the identifier provided on or associated with the medicament container and the short-range communication interface of the electronic unit of the injection device.

According to a further aspect, the present disclosure also relates to an electronic device. The electronic device includes a short-range communication interface operable to communicate with a short-range communication interface of an injection device as described above. The electronic device further includes a wireless reader interface directly or indirectly coupled to the short-range communication interface and operable to capture or to read the identifier provided on or associated with the medicament container of the injection device.

The electronic device may be implemented as a mobile or wearable electronic device. The electronic device may include a smart phone, a smart watch or a tablet computer. The electronic device may provide a relay for reading or capturing information of the identifier provided on or associated with the medicament container and being operable to transmit or forward this information to the short-range communication interface of the electronic unit of the injection device. The electronic device may unalterably transfer or transmit the information to the electronic unit of the injection device as gathered from identifier.

Here, the electronic unit of the injection device is inoperable to gather or to read information of the identifier. In some embodiments, the identifier of the medicament container is exclusively readable by a NFC interface having a limited transmission range, which is smaller than a spatial distance between the electronic unit of the injection device and the identifier when provided on one of the medicament container and the distal housing component and when the medicament container assembled inside the distal housing component is arranged inside the housing of the injection device.

In some embodiments, the identifier of the medicament container may be exclusively readable by the wireless reader interface of the electronic device.

Generally, the electronic device is configured to communicate and/or to cooperate with the injection device as described above. Insofar, all features, effects and benefits as described above in connection with the injection device equally apply to the electronic device.

According to a further example, the electronic device, in particular the wireless reader interface thereof includes at least one of a camera to capture an optical code of the identifier and a NFC interface to communicate with a passive electronic identifier, e.g., with a NFC tag of the identifier. In either way, the wireless reader interface of the external electronic device is operable to acquire, to capture, to read or to retrieve information of the identifier and to forward this information to the electronic unit of the injection device via the short-range communication link between the short-range communication interface of the electronic unit of the injection device and a corresponding short-range communication interface of the electronic device.

According to a further example, the electronic device includes a wide range communication interface configured to communicate with a database via a network. The wide range communication interface may be implemented as a mobile phone-based communication interface, such as USM, LTE, 4G, 5G, 6G or any other telecommunication standards. The wide range communication interface may be implemented to communicate in a wireless wide area network (WANW). Such networks are created through the use of mobile phone signals provided and maintained by specific mobile phone (cellular) service providers.

The short-range communication interface may be implemented as a wireless local area network (WLAN). Such networks are wireless networks that use radio waves. The backbone network usually uses cables, with one or more wireless access points connecting the wireless users to the wired network. The transmission range of such WLAN network can be anywhere from a single room to an entire campus. The short-range communication interface may be also implemented to communicate with a wireless personal area network (WPAN), such as a short-range network that uses Bluetooth or BLE. The short-range communication interface may provide a transmission range of several meters.

The NFC interface as described above uses a communication protocol for communication between two respective electronic devices over a distance of a few centimeters or less. In some embodiments the transmission range of the NFC interface is less than or equal to 4 cm, less than or equal to 3 cm, less than or equal to 2 cm. The NFC interface may be based on inductive coupling between two so-called antennas present on NFC-enabled devices communicating in one or both directions, using a frequency of 13.56 Megahertz (MHz) in the globally available unlicensed radio frequency ISM band using the ISO/IEC 18000-3 air interface standard at data rates ranging from 106 to 424 kilobits per second (kb/s).

With the electronic device including a wide range communication interface and/or by way of the short-range communication interface, the electronic device and/or the electronic unit of the injection device is configured to connect to a database via a network, such as the internet. In this way, a communication with a database is enabled. Here, a data synchronization can be provided. Data from the storage and/or data provided by the database can be compared and evaluated with data obtained from the identifier.

Here, the electronic device and/or the electronic unit may be implemented to transmit identifier related data to the database and to retrieve respective data from the database in return. This way, information about the identifier and hence information about the medicament container can be synchronized with the data provided in the database. Here, a data evaluation or data approval can be performed. For instance, and by way of the communication between the external electronic device and the database a checkup can be conducted e.g., if a unique identifier associated with the medicament container is a valid identifier or if such an identifier has been marked as invalid in the database.

In this way, each individual medicament container can be tracked and can be at least virtually marked in the database as being valid or invalid. Here, a valid identifier as stored in the external database can be marked or overwritten as invalid once the identifier has been detected by the external electronic device and through data communication between the external electronic device and the database via the network. This way, counterfeited containers can be precisely detected, and patient safety can be thus improved. In effect, only original or genuine medicament containers provided with a valid identifier can be used to unlock the drive mechanism of the injection device.

According to a further aspect, the present disclosure also relates to an injection system. The injection system includes an injection device as described above, which may be equipped with a medicament container. The injection system further includes an electronic device as described above. Insofar, any features, effects, and benefits as described above in connection with the injection device and/or in connection with the electronic device equally apply to the injection system; and vice versa.

According to an example, a distance between the identifier as provided on or in one of the distal housing component and the medicament container and the short-range communication interface of the injection device is larger than a transmission range of the NFC interface of the electronic device. This may particularly apply for examples or implementations of the identifier as a passive electronic identifier, in particular, when the identifier is implemented as an NFC tag. Here, the electronic unit of the injection device may be void of a corresponding NFC interface or NFC reader.

Even if the electronic unit is equipped with such an NFC reader, the spatial distance between such a reader of the electronic unit and the corresponding electronic identifier of the medicament container would exceed the transmission range of the NFC communication link. Hence, providing the electronic unit with a NFC reader would be difficult because when the identifier is provided in or on the medicament container or is provided in or on the distal housing component, the identifier is out of the transmission range of the NFC reader, e.g., provided at or near the proximal end of the proximal housing component.

Here, the external electronic device provides a relay function to communicate with the passive electronic identifier of the medicament container and the short-range communication interface of the electronic unit of the injection device.

According to another aspect, the present disclosure relates to a method of unlocking operation of an injection device for at least one of setting or injecting of a dose of a medicament. The injection device includes a housing configured to accommodate a medicament container and a drive mechanism. The drive mechanism is operably engageable with the medicament container for expelling or withdrawing the dose of the medicament from the medicament container. In some embodiments the drive mechanism includes a piston rod to exert a distally directed pressure onto a stopper movably arranged in a barrel of the medicament container.

The drive mechanism further includes at least one interlock configured to disable at least one of setting of the dose and dispensing of the dose. The method of unlocking operation of the injection device includes the step of capturing or reading of an identifier by a wireless reader interface of an external electronic device. The identifier is provided on or is associated with the medicament container. In some embodiments, the identifier is provided on the medicament container or is integrated into the medicament container. With other examples the medicament container may be preassembled in a distal housing component of the injection device. Here, the identifier may be attached to the distal housing component or may be integrated into the distal housing component.

The method further includes the step of establishing a communication link between a short-range communication interface of the injection device and a corresponding short-range communication interface of the electronic device. Furthermore, the method includes the step of transmitting information being indicative of the identifier and/or containing information about the identifier/or about the medicament container from the electronic device to the injection device to at least one of temporally deactivating or re-activating the at least one interlock.

Additionally, or alternatively, the method also includes the step of at least temporally deactivating the at least one interlock in order to support and/or to allow at least one of setting and dispensing of the dose of the medicament.

Generally, the method of unlocking operation of the injection device is to be implemented by an injection device in combination with an external electronic device, hence by way of an injection system, as described above. Insofar, all features, effects and benefits described above in connection with the injection device, the electronic device and the injection system equally apply to the method of unlocking operation of the injection device, and vice versa.

According to a further example, the method includes the step of validating the identifier by comparing medicament-related data obtained from the identifier with predefined data related to the injection device. In this way, it can be determined or asserted if the identifier matches with predefined data related to the injection device and if the medicament container is usable or is intended for use with the injection device.

If the acquired or captured identifier-related data does not match with predefined data related to the injection device, the drive mechanism is either actively locked or remains locked by the interlock. Then, the injection device is effectively inoperable to set a dose of the medicament and/or to inject a dose of the medicament. With further examples the method and the step of validating the identifier may include to modify the identifier. This may particularly apply through a NFC link between the electronic identifier and the external electronic device.

In some embodiments, the external electronic device may read out the electronic storage of the passive electronic identifier. Upon such a readout activity, a respective readout flag of the electronic identifier may be set so as to mark the electronic identifier as being currently in use or as having been used. A repeated use of the passive electronic identifier and/or of the medicament container associated with the identifier may be then effectively impeded or blocked, e.g., to prevent or to impede an unintended and/or unauthorized use of the medicament container. Insofar, capturing or reading of the identifier may include an evaluation or approval of the status of the readout flag provided by the electronic storage of the electronic identifier.

Alternatively, and with further examples, it is possible that upon readout of the identifier provided on or associated with the medicament container by the external electronic device there is invoked a database request with an external database, e.g., via a network connection. In the database, the medicament container currently in use may be marked to be ready for use or of not being suitable for use. With such a database request the external electronic device may gather additional information if a particular medicament container can be used or should not be used or cannot be used with the injection device.

Accordingly, the communication link between the external electronic device and the local electronic unit of the injection device may be used to control, e.g., to lock or to release the interlock so as to enable or to disable operation of the injection device with the given medicament container.

According to a further aspect, the present disclosure relates to a computer readable medium including instructions that, when executed by an electronic device, causes the electronic device to execute the steps of capturing or reading of an identifier provided on or being associated with the medicament container, to establish a communication link between a short-range communication interface of the injection device and the corresponding short-range communication interface of the electronic device. Furthermore, the computer readable medium causes the electronic device to execute the step of transmitting information indicative of the identifier from the electronic device to the injection device to at least temporarily deactivate the at least one interlock.

The computer readable medium is particularly configured to execute the steps of the above-mentioned method of unlocking operation of the injection device. Insofar, all features, effects and benefits as described above with the method of unlocking also apply to the computer-readable medium, mutatis mutandis.

According to a further aspect, the disclosure relates to a computer readable medium including instructions which, when executed by a processor of the electronic unit of the injection device, cause the processor to control the interlock of the drive mechanism of the injection device. The computer readable medium may include instructions, which, when executed by the processor of the electronic unit cause the electronic unit to process data obtained from the external electronic device and to lock or to unlock the at least one interlock on the basis of the data or signals obtained from the external electronic device. The computer readable medium is particularly executable by the electronic unit of the injection device. Insofar, all features, effects and benefits as described above in connection with the injection device equally apply to the respective computer readable medium; and vice versa.

Generally, the scope of the present disclosure is defined by the content of the claims. The injection device is not limited to specific embodiments or examples, but includes any combination of elements of different embodiments or examples. Insofar, the present disclosure covers any combination of claims and any technically feasible combination of the features disclosed in connection with different examples or embodiments.

In the present context, the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded

DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codable amino acids, or amino acids, including non-codable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega -carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ωcarboxyheptadecanoyl)-de(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929,ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin,

Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., the antibody has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full-length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti-IL-6 mAb (e.g., Sarilumab), and anti-IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

FIG. 1 shows a longitudinal cross-section of one example of an injection device.

FIG. 2 shows the injection device according to FIG. 1, with the distal housing component disconnected from the proximal housing component.

FIG. 3 shows a longitudinal cross-section through an example of a drive mechanism implemented with the injection device according to FIG. 1.

FIG. 4 shows the interaction between an electronic unit of the injection device according to FIG. 1 and an external electronic device.

FIG. 5 shows a block diagram of an injection system, including the injection device according to FIG. 1 and the external electronic device according to FIG. 4.

FIG. 6 shows a distal housing component provided with an identifier.

FIG. 7 shows the distal housing component according to FIG. 6 and a medicament container provided with the identifier.

FIG. 8 shows an example of an electronic identifier.

FIG. 9 shows an example of an optically implemented identifier.

FIG. 10 shows a flowchart of a method of unlocking operation of the injection device according to FIG. 1.

### DETAILED DESCRIPTION

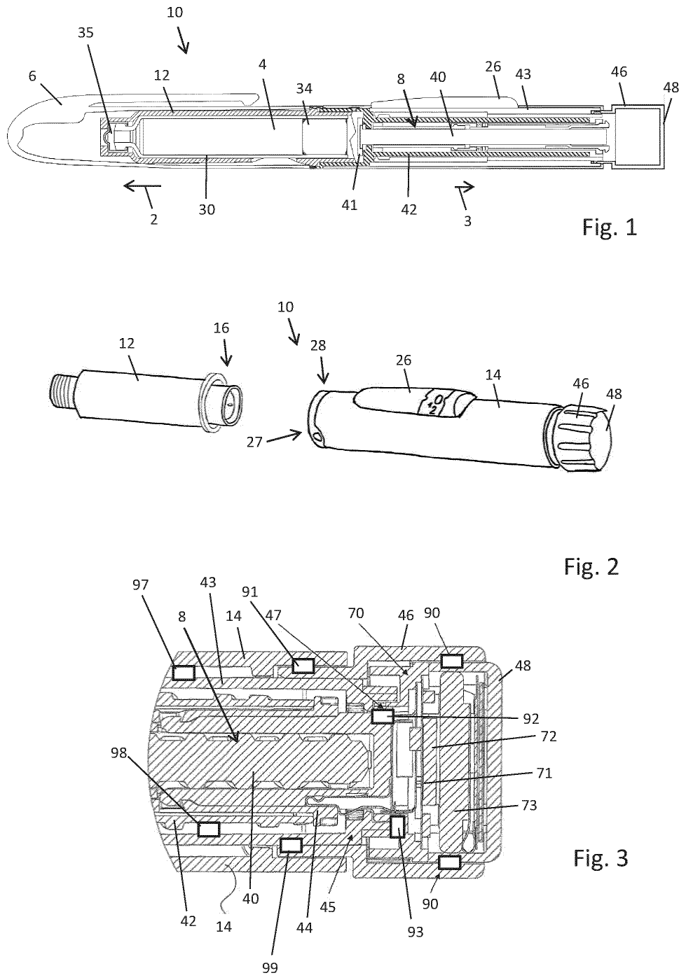

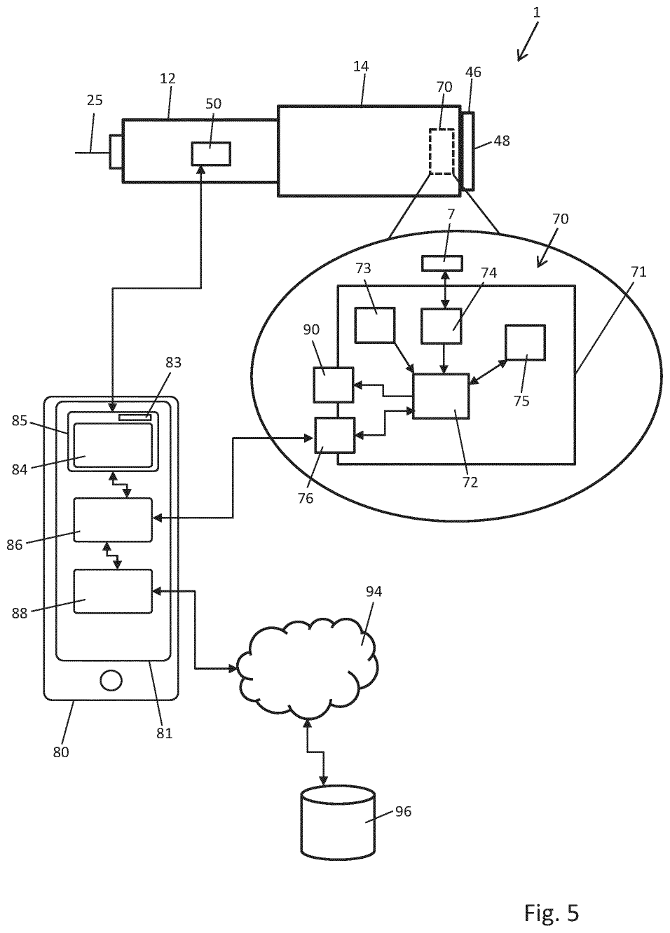

FIG. 1 shows an example of an injection device 10 implemented as a pen-type injector. The injection device 10 is part of an injection system or drug delivery system 1, as illustrated in FIG. 5. The injection device 10 includes a housing 11. The housing 11 may be of elongated or tubular shape. The housing 11 extends in a longitudinal or axial direction. Towards the distal direction 2 the injection device 10 includes a protective cap 6 covering a distal housing component 12. The housing 11 further includes a proximal housing component 14 detachably connectable with the distal housing component 12. As described with reference to FIGS. 1, 2 and 6, the proximal housing component 14 includes a connecting end towards the distal direction 2.

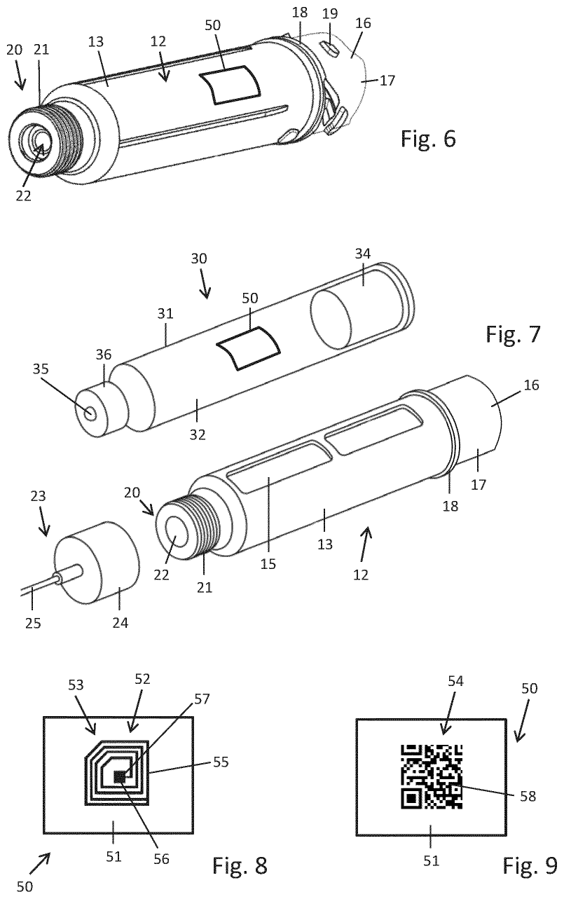

The connecting end of the proximal housing component 14 includes a receptacle 27 configured to receive a complementary-shaped proximal connecting end 16 of the distal housing component 12. As illustrated in FIGS. 2 and 6, the proximal connecting end 16 of the distal housing component 12 includes an insert portion 17 configured for connection with a distal connecting end 28 of the proximal housing component 14. For this, the proximal housing component 14 includes a receptacle 27 at the distal connecting end 28 configured and shaped to receive the insert portion 17 therein.

On an outside surface of the insert portion 17 there is provided at least one fastening element 19, e.g., implemented as a protrusion to mate or to engage with a complementary-shaped recess on an inside surface of a sidewall of the receptacle 27. The insert portion 17 and the receptacle 27 may include mutually corresponding or complementary shaped fastening elements, so as to form a snap fit engagement, a bayonet joint or a threaded joint.

The insert portion 17 is delimited in longitudinal direction by a radially outwardly extending flange 18. When the housing components are fully assembled the flange 18 is in proximal longitudinal abutment with a distal end face of a sidewall or of a distal end of the proximal housing component 14.

The distal housing component 12 is implemented as a cartridge holder. The distal housing component 12 includes a tubular-shaped sidewall 13 configured and sized to receive a medicament container 30 as illustrated in FIG. 7. The cartridge holder as shown in FIG. 7 includes at least one aperture 15 in the tubular-shaped sidewall 13. Towards the distal end the tubular-shaped sidewall 13 radially narrows and forms a shoulder portion adjoining into a distal connecting end 20. The medicament container 30 is typically filled with an injectable medicament 4, e.g., provided or stored in liquid form inside the medicament container 30.

The connecting end 20 includes a mount 21 for a needle assembly 23. The needle assembly 23 includes a needle hub 24 configured for detachable or releasable engagement with the mount 21 and further including an injection needle 25. The injection needle 25 can be implemented as a double-tipped injection needle. When correctly assembled on the mount 21 a proximal tipped end of the injection needle 25 reaches through a distal through opening 22 at the connecting and 20 and pierces a seal 35 provided at the distal end of the medicament container 30 located inside the cartridge holder.

The seal 35 may be implemented as a rubber disc and may serve as a pierceable septum. The seal 35 may be mechanically fixed to the distal end of the barrel 31 of the medicament container 30 by way of a crimped metal cap 36. Towards the proximal end the tubular shaped barrel 31 of the container 30 is sealed by a stopper 34, e.g., implemented as a rubber stopper. The piston or stopper 34 is movably disposed inside the barrel 31. The piston or stopper 34 may be displacable towards the distal direction 2, hence towards the dispensing end of the injection device 10 through the action of a piston rod 40 of a drive mechanism 8 provided and assembled inside the proximal housing component 14.

The drive mechanism 8 can be implemented in different ways. Insofar the present disclosure is by no way limited to the presently illustrated example of a particular drive mechanism 8. In the presently illustrated example, the drive mechanism 8 includes a piston rod 40 equipped with a pressure piece 41 at the distal end. The pressure piece 41 is configured to apply distally directed pressure onto the stopper 34 of the medicament container 30. The drive mechanism or the injection device further includes a housing insert 42, which serves as a mount for numerous movable components of the drive mechanism 8.

In some embodiments, the drive mechanism 8 includes a number sleeve 43 provided with a consecutive sequence of numbers on an outside facing sidewall. These numbers are visible through a window 26 or through a respective aperture in the sidewall of the proximal housing component 14. The drive mechanism 8 further includes a driver 44, which may be implemented as a drive sleeve. For setting of a dose, the number sleeve 43 may be subject to a rotation relative to the housing 11 along a first sense of rotation. During dispensing or injecting of the dose the number sleeve 43 is subject to a rotation along a second and hence opposite sense of rotation.

The driver 44 or drive sleeve can be permanently or non-permanently operably engaged with the piston rod 40. At least during dispensing of a dose, the driver 44 is in a force-or torque-transmitting engagement with the piston rod so as to urge the piston rod 40 in distal direction 2 for dose expelling or dose injecting. With some implementations of the drive mechanism 8, the driver 44 is subject to a rotation along the second sense of rotation during dose dispensing. In some embodiments, the driver 44 is subject to a rotation along the first sense of rotation during and/or for setting of the dose. In some embodiments, the driver 44 is rotationally and/or axially fixed relative to the housing 11 during setting of the dose but is movable during dispensing or injecting of the dose.

The drive mechanism 8 further includes a dose dial 46, e.g., provided at the proximal end of the proximal housing component 14. The dose dial 46 provides a user grippable or user operable tool for setting of the dose. In some embodiments the dose dial 46 is rotatable by a user relative to the housing 11 for setting of the dose. The drive mechanism 8 further includes a trigger 48, e.g., implemented as a dose button depressible by a user in distal direction 2 for initiating and/or for controlling the process of dose dispensing or dose injection.

The trigger 48 and the dose dial 46 may be provided on a common part. They may be integrally formed. They may be both located on a so-called dial extension of the injection device 10, which is subject to a helical motion relative to the housing 11 during setting of the dose and which is subject to a longitudinal distally directed movement during and/or for dispensing or injecting of the dose. In some embodiments, the trigger 48 is provided at a proximal end of the dose dial 46. The trigger 48 may form or constitute a proximal end face of the housing 11.

In the example of FIG. 3, there are indicated numerous possible implementations of an interlock 90, 91, 92, 93, 97, 98, 99. Here, the interlock 90 is provided as an interlock between the trigger 48 and the dose dial 46. With an activated interlock 90 a mutual displacement of the trigger 48 relative to the dose dial 46 can be effectively blocked. When the trigger 48 is non-rotationally fastened to the housing 11 or to the dial extension an activated interlock 90 may also block a rotation of the dose dial 46 relative to the trigger 48 and hence relative to the housing 11. In this way, dispensing of a dose and/or dialing or setting of a dose can be effectively blocked or deactivated by activation of the interlock 90.

The interlock 91 is in mechanical connection with the proximal housing component 14 and with at least one of the dose dial 46, the trigger 48 and the dial extension. Activation of the interlock 91 serves to block or to impede at least one of a rotation and a longitudinal sliding movement of the dose dial 46 relative to the housing component 14. With an activated interlock 91 setting of a dose and/or dispensing of the dose can be effectively blocked.

The drive mechanism 8 may further include at least a first clutch 45 and a second clutch 47. By way of the clutches 45, 47 the drive mechanism 8 is switchable between a dose setting mode and a dose injecting mode. Depending on the specific implementation of the drive mechanism 8, the clutches 45, 47 are operable to mutually engage any two of the following components:

piston rod 40, housing insert 42, housing 11, number sleeve 43, driver 44, dose dial 46 and/or trigger 48. Details of one example of a drive mechanism are further described in document WO 2014/033195 A1. The clutch 45 may be provided with a further interlock 93 and the clutch 47 may be provided with the interlock 92. The interlock 92 may be operably engaged with the clutch 47. Likewise, the interlock 93 may be operably engaged with the clutch 45.

With an activated interlock 92, 93, operation of the respective clutch 45, 47 may be blocked so as to impede a switching of the drive mechanism 8 from the dose setting mode into the dose injecting mode and/or vice versa, namely from the dose injecting mode into the dose setting mode.

With a further example as illustrated in FIG. 3 the number sleeve 43 is selectively engageable with the proximal housing component 14 via an interlock 97. With a further example the number sleeve 43 is selectively engageable with the housing insert 42 via an interlock 98. With a further example as illustrated in FIG. 3 the number sleeve 43 is selectively engageable with at least one of the dose dial 46 and the trigger 48 via another interlock 99.

By way of the interlock 97 at least one of a rotation and a longitudinal movement of the number sleeve 43 relative to the proximal housing component 14 can be blocked or impeded. By way of the interlock 98 at least one of a rotation and a longitudinal movement of the number sleeve 43 relative to the housing insert 42 can be selectively blocked or impeded. Since the number sleeve 43 is subject to at least one of a rotational, helical, or longitudinal sliding movement relative to the proximal housing component 14 during setting of the dose and injecting of the dose a respective blocking of a movement of the number sleeve 43 serves to block or to impede at least one of a dose setting and a dose injecting action.

By way of the interlock 99 a movement, e.g., a rotational and/or longitudinal movement of the dose dial 46 and/or of the trigger 48 can be blocked relative to the number sleeve 43. This way and with typical implementations of the drive mechanism 8, at least injecting of a dose can be effectively impeded or prevented when the interlock 99 is activated.

With typical implementations the interlocks 90, 91, 92, 93, 97, 98, 99 each include an electromechanical actuator and at least one of a latch and a clamp. This way, the latch or clamp is movable between a locking configuration and a release configuration by the actuator, wherein operation of the actuator is controllable by electrical signals, e.g., obtained from or generated by a processor 72 the function of which will be described below. In some embodiments, the latch provided on a first component of the injection device 10 is configured to engage with a second component of the injection device in a form fitting manner.

With other examples and wherein the interlock includes a movable clamp, the clamp provided on a first component of the injection device is configured to engage with a second component of the injection device in a friction fitting manner. In either way and by activation of the interlock 90, 91, 92, 93, 97, 98, 99 there can be either provided at least one of a form fitting engagement and a frictional engagement between the first and the second component of the injection device so as to block or to impede at least one of setting of a dose and dispensing of a dose.

The actuator may include at least one of an electromagnet and an electromechanical drive controllable by the processor 72. By way of the actuator the latch, e.g., implemented as a movable or elastically deformable bending arm, hook or clamp is movable between a locking position, in which the interlock 90, 91, 92, 93, 97, 98, 99 is activated, and a release position, in which the interlock 90, 91, 92, 93, 97, 98, 99 is deactivated. In the deactivated configuration, the interlock 90, and 91, 92, 93, 97, 98, 99 allows and supports a movement of a first component relative to a second component of the drive mechanism so as to enable at least one of setting of a dose and injecting of the dose.

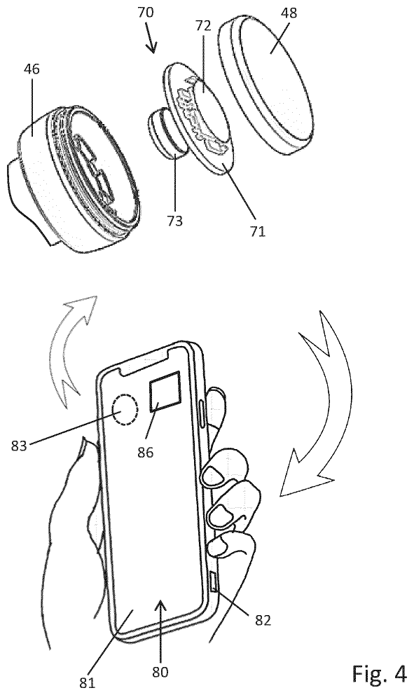

The injection device 10 is further provided with an electronic unit 70. In the present example, the electronic unit 70 is embedded inside the injection device 10. The electronic unit 70 is arranged at or near the proximal end of the housing 11. The electronic unit 70 may be located or arranged inside a dial extension of the injection device 10. As illustrated in FIGS. 3 and 4, the electronic unit 70 is arranged inside a hollow space of the cylindrically shaped dose dial 46, which is covered and/or closed towards the proximal direction 3 by the trigger 48. Here, the trigger 48 provides an end cap forming a proximal closure of the hollow dose dial 46. In some embodiments, the trigger 48 and the dose dial 46 are integrally formed.

The electronic unit 70 includes a printed circuit board 71 provided with a processor 72. The electronic unit 70 is further provided with a source of electric energy, which can be implemented as an electric battery 73. As illustrated, the processor 72 and the battery 73 may be provided on opposite sides of the printed circuit board 71.

The electronic unit 70 is schematically illustrated in the block diagram of FIG. 5. The electronic unit 70 further includes at least a short-range communication interface 76 operable to communicate with a corresponding short-range communication interface 86 of an external electronic device 80. The processor 72 is connected to the interlock 90 and is operable to control operation of the interlock 90. The interlock 90 may be implemented as an electromechanical interlock. The interlock 90 may include a latch or any other type of a mechanically movable component so as to block or to impede operation of the drive mechanism 8.

In some embodiments, the latch or the movable component of the interlock 90 is movable through and by an actuator that is electronically controllable by the processor 72. By way of the short-range communication interface 76 the electronic unit 70 is operable to communicate with the external electronic device 80. Optionally, the electronic unit 70 is provided with a sensor 74 operable to detect or to measure movement of at least one movable component 7 of the drive mechanism 8. The movable component 7 may be represented by any of the above-mentioned movable components, e.g., the piston rod 40, the number sleeve 43, the driver 44, the trigger 48, the dose dial 46 and/or any of the clutches 45, 47. By way of the sensor 74, which may be implemented as one of an optical sensor, a capacitive sensor, an inductive sensor, an optical sensor or as an acoustic sensor, operation of the drive mechanism 8 and hence operation of the injection device 10 can be supervised and monitored.

By way of the sensor 74, information regarding the size of the dose currently set, dialed or dispensed can be obtained and can be stored in a local storage 75. Data repeatedly captured or obtained during repeated and subsequent use of the injection device 10 can be stored in the storage 75. Such injection-related data stored in the storage 75 can be synchronized by the short-range communication interface 76 with the external electronic device 80 and/or with an external database 96, e.g., hosted or provided by a healthcare provider.

The external electronic device 80 includes a wireless reader interface 85 configured to read or to capture an identifier 50 provided on or associated with the medicament container 30. By way of the external electronic device 80, and in particular by way of the wireless reader interface 85, an information content or data of the identifier 50 can be detected or read out by the external electronic device 80. Information gathered by the wireless reader interface 85 can be transmitted to the electronic unit 70 of the injection device via a communication link between the short-range communication interface 76 of the electronic unit 70 and the corresponding short-range communication interface 86 of the external electronic device 80.

In some embodiments the external electronic device 80 is implemented as a smart phone, as a smart watch or as a tablet computer. The electronic device 80 may include a wide range communication interface 88 to establish a communication link to the external database 96 by a communication network 94. In some embodiments the external electronic device 80 is a mobile device or wearable device. The electronic device 80 includes a display 81 and/or a speaker 82 to communicate with a user.

The wireless reader interface 85 may include a NFC interface 84 and/or a camera 83. The camera 83 may be operable to read the identifier 50 when implemented as an optical identifier 54 as illustrated in FIG. 9. The optical identifier 54 may include a one or two-dimensional optical code 58, e.g., a QR code or data matrix code capturable by the camera 83 of the external electronic device 80.

In the example of FIG. 8, the identifier 50 is implemented as a passive electronic identifier 52. Here, the identifier 50 may include a NFC tag 53 provided with an antenna 55 and with a microchip 56. The microchip 56 may include a local storage or memory 57, e.g., to store medicament related information and/or or to store medicament container related information. The memory 57 can be implemented as a read-only memory or a re-writable memory.

In either way, the identifier 50 may include a planar substrate 51 for attaching the identifier 52 a sidewall 13 of the distal housing component 12 or to a sidewall of the barrel 31 of the medicament container 30. The planar substrate 51 may include a pliable or deformable sheet structure with an adhesive layer to adhesively attached the substrate 51 and hence the identifier 50 to the housing component 12 or to the medicament container 30.

The identifier 50, implemented as optical identifier 54 or as passive electronic identifier 52 can be non-detachably provided on or in the medicament container 30 and/or on or in the distal housing component 12. The information or data stored or provided by the identifier 50 may be indicative of a type or name of the medicament, a medicament manufacturing date, a medicament expiry date, a LOT number of the medicament and/or of a unique medicament container ID. The data or information provided by the identifier 50 can be acquired, retrieved, captured, or obtained via the wireless reader interface 85 of the external electronic device 80.

The respective information can be either processed or pre-processed by the external electronic device 80 or can be simply forwarded or transmitted to the electronic unit 70 by the communication link between the short-range communication interface 76 and the corresponding short-range communication interface 86. This way, a respective processing of the data obtained and retrieved from the identifier 50 can be also conducted by the processor 72 of the electronic unit 70. Here, the external electronic device 80 may simply provide a relay or may function as a relay. This way, data or information provided by or stored by the identifier 50 can be provided to the electronic unit 70, which itself is inoperable to read or to capture the data or information provided by the identifier 50.

This approach helps avoid implementation of a NFC interface for the electronic unit 70. This approach is sufficient when the electronic unit 70 is equipped with one communication interface, namely with a short-range communication interface 76, e.g., for data or information exchange with the external electronic device 80. The external electronic device 80 may be readily equipped with two different wireless communication interfaces, namely with the short-range communication interface 86, e.g., implemented as a Bluetooth or Wi-Fi interface to communicate with the short-range communication interface 76 of the electronic unit 70. The electronic device 80 may be readily equipped with a further communication interface implemented as an NFC interface 84 to provide reading or capturing of data or information as provided by the identifier 50.

When implemented as a passive electronic identifier 52, e.g., as an NFC tag 53, the external electronic device 80 is brought in close vicinity to the identifier 50, which may be located inside the housing 11 of the injection device 10. In some embodiments, the transmission range of the NFC interface 84 is a few centimeters. Upon establishing a communication link, e.g., upon reading of the passive electronic identifier 52 by the NFC interface 84, the external electronic device 80 may provide an acoustic or visual confirmation to the user. By the display 81 and/or by the speaker 82 the user may be informed that the electronic identifier 52 has been successfully read out.

The external electronic device 80 may be wirelessly paired to the electronic unit 70. Upon successful transferring of the data or information of the identifier 50 to the electronic unit 70 via the communication link between the short-range communication interfaces 86, 76 another visual and/or audible confirmation may be provided to the user, e.g., by the electronic unit 70 and/or by the external electronic device 80.

If approval or processing of the data or information of the identifier 50 as obtained via the short-range communication interface 76 reveals that the medicament container 30 is intended for use with the injection device 10, the processor 72 is operable to deactivate the interlock 90, thereby allowing to set and/or to dispense or inject a dose of the medicament.

The processor 72 and/or the at least one interlock 90, 91, 92, 93 may be implemented in various different ways. With one approach, the interlock 90 or locking mechanism may be operable to lock setting of a dose, to lock dispensing of a dose order to lock both, setting and dispensing of a dose.

The interlock 90 and/or the processor 72 may be further configured to implement an automatic re-locking, e.g., after termination of each dispensing or in injecting procedure. Moreover, and based on signals obtainable from the sensor 74 the processor 72 may be provided with information being indicative of the remaining filling level of the medicament container 30. In this way, when the sensor 74 provides respective sensor signals that the medicament container 30 is substantially depleted or empty, the processor 72 may invoke activation of the interlock 92. Removal of the medicament container 30 or insertion of the medicament container 30, as well as disconnection or reconnection of the housing components 12, 14 may trigger an automated locking or disabling of the function of the drive mechanism for setting and/or for dispensing of a dose.

Information provided by the identifier 50 and capturable by the wireless reader interface 85 may also include a counterfeit protection. Here, a unique code needs to be verified, either by the electronic unit 70 and/or by a data exchange or data approval with the external database 96.

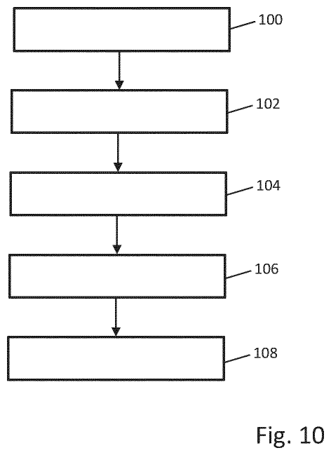

In the example of FIG. 10, in a first step 100 a user may bring the external electronic device 80 in close vicinity to the injection device 10. In a subsequent step 102, the wireless reader interface 85 captures or reads the identifier 50 as provided on one of the distal housing component 12 and the medicament container 30. In a further step 104 the respective information captured by the wireless reader interface 85 is transmitted to the electronic unit 70 of the injection device 10 via the short-range communication interfaces 76, 86.

In the subsequent step 106 the processor 72 performs a data check and evaluates or processes the data obtained from the short-range communication interface 76. In case that the medicament container 30 is intended for use with this particular injection device 10 the respective data is approved and in step 108 the interlock 90 is controlled or operated by the processor 72 to unlock the drive mechanism 8 of the injection device. A user may then commence to use the injection device 10 for setting of a dose and/or for injecting of a dose of the medicament 4. With other situations, and when the medicament container 30 is not intended for use with the injection device 10, the interlock 90 is triggered to block or to lock the drive mechanism 8 of the injection device 10.

### REFERENCE NUMBERS

1 drug delivery system
2 distal direction
3 proximal direction
4 medicament
6 protective cap
7 movable component
8 drive mechanism
10 injection device
11 housing
12 housing component
13 sidewall
14 housing component
15 aperture
16 connecting end
17 insert portion
18 flange
19 fastening element
20 connecting end
21 mount
22 through opening
23 needle assembly
24 needle hub
25 injection needle
26 window
27 receptacle
28 connecting end
30 container
31 barrel
32 sidewall
34 stopper
35 seal
36 cap
40 piston rod
41 pressure piece
42 housing insert
43 number sleeve
44 driver
45 clutch
46 dose dial
47 clutch
48 trigger
50 identifier
51 substrate
52 electronic identifier
53 NFC tag
54 optical identifier
55 antenna
56 chip
57 memory
58 code
70 electronic unit
71 printed circuit board
72 processor
73 battery
74 sensor
75 storage
76 short range communication interface
80 external electronic device
81 display
82 speaker
83 camera
84 near field communication interface
85 wireless reader interface
86 short range communication interface
88 wide range communication interface
90 interlock
91 interlock
92 interlock
93 interlock
94 network
96 database

1-17. (canceled)

18. An injection device for injecting of a dose of a medicament, the injection device comprising:
a housing configured to accommodate a medicament container;
a drive mechanism operably engageable with the medicament container for expelling or withdrawing the dose of the medicament from the medicament container, wherein the drive mechanism comprises at least one interlock configured to disable at least one of setting of the dose or dispensing of the dose;
an identifier provided on or associated with the medicament container;
an electronic unit operable to control the at least one interlock, wherein the electronic unit comprises a short range communication interface to communicate with a corresponding short range communication interface of an external electronic device for obtaining information indicative of the identifier, and wherein the electronic unit is configured to process signals of the short range communication interface to at least temporarily deactivate the at least one interlock.

19. The injection device according to claim 18, wherein:
the housing comprises a distal housing component and a proximal housing component,
the distal housing component is configured to accommodate the medicament container, and
the proximal housing component is configured to accommodate or to support the drive mechanism.

20. The injection device according to claim 19, wherein:
the identifier is located on or inside the distal housing component, and
the electronic unit is provided in or on the proximal housing component.

21. The injection device according to claim 18, wherein the identifier comprises at least one of an optical code and a visible code capturable by the external electronic device.

22. The injection device according to claim 18, wherein the identifier comprises a passive electronic identifier.

23. The injection device according to claim 22, wherein the passive electronic identifier comprises a near-field communication (NFC) tag configured to communicate with a corresponding NFC interface of the external electronic device.

24. The injection device according to claim 23, wherein the electronic unit of the injection device is free of a corresponding NFC interface or NFC reader.

25. The injection device according to claim 18, wherein the short-range communication interface comprises a wireless communication interface based on one of the following communication standards: Bluetooth, Bluetooth low energy (BLE), Zigbee, or Wi-Fi.

26. The injection device according to claim 18, wherein:
the at least one interlock comprises an electromechanical interlock configured to mechanically engage with a first component of the injection device and with a second component of the injection device,
the first component is movable relative to the second component for at least one of setting of the dose or injecting of the dose, and
the at least one interlock is operable to switch between an interlocked state in which the first component is movably locked to the second component, and a release state in which the first component is movable relative to the second component.

27. The injection device according to claim 18, wherein:
the drive mechanism comprises at least one clutch operable to switch the drive mechanism between a dose setting mode and a dose injecting mode, and
the at least one interlock is operably engaged with the at least one clutch to selectively disable switching of the drive mechanism from one of the dose setting mode and the dose injecting mode into the other one of the dose setting mode and the dose injecting mode.

28. The injection device according to claim 18, wherein the electronic unit is inoperable to gather or to read information of the identifier.

29. An electronic device comprising:
a short-range communication interface operable to communicate with a short-range communication interface of an injection device, and
a wireless reader interface coupled to the short-range communication interface and operable to capture or to read an identifier provided on or associated with a medicament container of the injection device.

30. The electronic device according to claim 29, wherein the wireless reader interface comprises at least one of:
a camera to capture an optical code of the identifier, and
an NFC interface to communicate with a passive electronic identifier of the identifier.

31. The electronic device according to claim 29, further comprising a wide-range communication interface configured to communicate with a database via a network.

32. An injection system comprising an injection device and an electronic device, the injection device comprising:
a housing configured to accommodate a medicament container;
a drive mechanism operably engageable with the medicament container for expelling or withdrawing the dose of the medicament from the medicament container, wherein the drive mechanism comprises at least one interlock configured to disable at least one of setting of the dose or dispensing of the dose;
an identifier provided on or associated with the medicament container;
an electronic unit operable to control the at least one interlock, wherein the electronic unit comprises a short range communication interface to communicate with a corresponding short range communication interface of an external electronic device for obtaining information indicative of the identifier, and wherein the electronic unit is configured to process signals of the short range communication interface to at least temporarily deactivate the at least one interlock; and the electronic device comprising:
a short-range communication interface operable to communicate with a short-range communication interface of the injection device, and
a wireless reader interface coupled to the short-range communication interface and operable to capture or to read the identifier provided on or associated with the medicament container of the injection device.

33. A method of unlocking operation of an injection device for at least one of setting or injecting of a dose of a medicament, wherein:
the injection device comprises a housing configured to accommodate a medicament container and a drive mechanism operably engageable with the medicament container for expelling or withdrawing the dose of the medicament from the medicament container, and
the drive mechanism comprises at least one interlock configured to disable at least one of setting of the dose and dispensing of the dose, the method comprising:
capturing or reading an identifier with a wireless reader interface of an external electronic device, wherein the identifier is provided on or is associated with the medicament container,
establishing a communication link between a short-range communication interface of the injection device and a corresponding short range communication interface of the electronic device, and
transmitting information indicative of the identifier from the electronic device to the injection device to at least temporarily deactivate or re-activate the at least one interlock.

34. The method according to claim 33, wherein:
the housing comprises a distal housing component and a proximal housing component,
the distal housing component is configured to accommodate the medicament container, and
the proximal housing component is configured to accommodate or to support the drive mechanism.

35. The method according to claim 34, wherein:
the identifier is located on or inside the distal housing component, and
the electronic unit is provided in or on the proximal housing component.

36. The method according to claim 33, wherein the identifier comprises at least one of an optical code and a visible code capturable by the external electronic device.

37. The method according to claim 33, wherein the identifier comprises a passive electronic identifier.
